(19) United States
(12) Patent Application Publication
(10) Pub. No.: US 2014/0271794 A1
(43) Pub. Date: Sep. 18, 2014

(54) ANTIMICROBIAL FOAM MEDICAL PATCH AND METHOD OF MAKING THE SAME

(71) Applicant: Patrick E. Eddy, Allendale, MI (US)

(72) Inventor: Patrick E. Eddy, Allendale, MI (US)

(21) Appl. No.: 14/216,029

(22) Filed: Mar. 17, 2014

Related U.S. Application Data
(60) Provisional application No. 61/788,647, filed on Mar. 15, 2013.

(51) Int. Cl. A61L 15/42; A61L 15/44

(52) U.S. Cl. CPC A61L 15/425; A61L 15/44
USPC 424/443; 514/63

(57) ABSTRACT

A foam medical patch is provided including a foam substrate having integrated therein an antimicrobial material comprising a silane quaternary ammonium salt. The antimicrobial material is substantially free from arsenic, silver, tin, heavy metals and polychlorinated phenols. The silane quaternary ammonium salt includes 3-(trimethoxysilyl) propyldimethyloctadecyl ammonium chloride.

### CROSS-REFERENCE TO RELATED APPLICATION

This application claims priority under 35 U.S.C. §119(e) upon U.S. Provisional Patent

Application No. 61/788,647, entitled “ANTIMICROBIAL FOAM MEDICAL PATCH AND METHOD OF MAKING THE SAME” filed on Mar. 15, 2013, by Patrick E. Eddy, the entire disclosure of which is incorporated herein by reference.

### BACKGROUND OF THE INVENTION

The embodiments described herein generally relate to a foam medical patch for medical applications and to a method of making such a foam medical patch.

### SUMMARY OF THE INVENTION

According to an embodiment of the present invention, a foam medical patch is provided. The foam medical patch comprises a foam substrate having integrated therein an antimicrobial material comprising a silane quaternary ammonium salt.

According to another embodiment of the present invention, a method is provided for making a foam medical patch, the method comprises providing a foam slurry including therein an antimicrobial material comprising a silane quaternary ammonium salt, and processing the slurry to form the foam medical patch.

According to another embodiment of the present invention, a method is provided for making a foam medical patch, the method comprises providing a foam medical patch, and treating the outer surfaces of the foam medical patch with an antimicrobial material comprising a silane quaternary ammonium salt.

In these three embodiments, the silane quaternary ammonium salt may comprise 3-(trimethoxysilyl) propyldimethyloctadecyl ammonium chloride.

These and other features, advantages, and objects of the present invention will be further understood and appreciated by those skilled in the art by reference to the following specification and drawings.

### DETAILED DESCRIPTION OF THE EMBODIMENTS

Reference will now be made in detail to the present preferred embodiments of the invention.

As noted above, the embodiments described herein pertain to foam medical patches.

Foam medical patches are used for various medical applications including surgical sponges and wound dressings. Such wound dressings may take a number of forms including bandages made of a rayon adhesive strip with a foam pad, as well as negative pressure wound therapy (NPWT) dressings. Because such foam medical patches are placed directly on or within wounds, it is important that they do not harbor microbes.

A novel foam medical patch is disclosed herein that not only provides the requisite properties for a medical-grade foam medical patch, but also eliminates bacteria on contact.

In general, foams are formed of slurries processed at relatively low temperatures. The foam slurry is made of a polymer, typically polyurethane, which is provided in a liquid form. The foam slurry is then reacted with water or some other substance, which causes the slurry to foam. The foam solidifies into a form that may then be used commercially. The low temperatures at which the foam slurry is formed allow an antimicrobial material to be mixed in with the slurry and therefore integrated within the resulting foam. Thus, for example, a foam medical patch may be made with a foam substrate and an antimicrobial material integrated throughout the foam substrate.

The antimicrobial material may be a silane quaternary ammonium salt. Preferred commercially available silane quaternary ammonium salts include: MicrobeCare™ XLP, which is available from MicrobeCare, LLC of Allendale, Mich.; “PROMOFRESH X 105” from Piedmont Chemical Industries I, LLC of High Point, N.C.; and AEM 5772 Antimicrobial, which is available from Aegis Environments of Midland, Mich. The antimicrobial could also be AEGIS Microbe Shield™ (from Aegis Environments, Midland, Mich.), which is a copolymer of chloropropyltrihydroxysilane and octadecylaminodimethyltrihydroxysilylpropyl ammonium chloride.

Silane quaternary ammonium salts are particularly well-suited for the antimicrobial material as they are long lasting and capable of emitting ions that aid in the destruction of a microbe. In addition, they are organofunctional silanes that include a monomer including a silane, a positively charged nitrogen molecule, and a long molecular chain. The silane bases of these monomers can covalently and permanently bond to each other and any surface. In addition, silane quaternary ammonium salts are preferable as they are substantially free from arsenic, silver, tin, heavy metals and polychlorinated phenols. Also, they are safe to apply to the skin or to a wound of a patient.

Microbes may include bacteria, mold, mildew, algae, etc. The cell membranes of the microbes are attracted to, and then are punctured by, the long molecular chains of the monomers. As the microbes are drawn closer because of the positive-negative ion exchanges, the monomers penetrate further into the cell membranes. Once the cell membranes are penetrated deeply, they are physically ruptured by a sword-like action and then electrocuted by positively charged nitrogen molecules of the monomers, thus destroying the microbes. Thus, the microbes are eliminated without “using up” any of the antimicrobial active ingredients, which remain in the foam ready to continue protecting the patient against further microbial contamination.

If the antimicrobial substance is a 3-(trimethoxysilyl) propyldimethyloctadecyl ammonium chloride material available from MicrobeCare, LLC, it includes an active ingredient of about 43 wt % 3-(trimethoxysilyl) propyldimethyloctadecyl ammonium chloride and about 57 wt % inert ingredients.

The antimicrobial substance may then be mixed into the foam slurry in various amounts of anywhere from about 0.01% to about 50% by weight to achieve the desired degree of antimicrobial activity while not compromising the desirable properties of the foam in the resulting mixture.

The foam slurry may consist of any conventional materials used for preparing a foam, particularly a urethane foam, other foams that may be used include polyethylene, Neoprene, vinyl, silicone and PVC foams.

The inventive foam imparts a durable antimicrobial finish to the surface and or cross sections of a wide variety of substrates. It is leach resistant and non-migrating and is not consumed by microorganisms. Some of the other benefits include: designed specifically with a solvent system to be easily dispersed into a urethane foam application; effectively inhibits the growth of mold and mildew, algae and bacteria on various surfaces; protects against microbial deterioration, discoloration and odor; provides an invisible, durable micro biostatic finish; and provides anti-static properties to surfaces.

While it is generally desirable to integrate the antimicrobial substance throughout the foam using the method disclosed above, some of the above benefits may be obtained (albeit to a lesser degree in some cases) by applying the antimicrobial substance to the outer surfaces of the foam after the foam has been formed.

Modifications of the invention will occur to those skilled in the art and to those who make or use the invention. Therefore, it is understood that the embodiments described above are merely for illustrative purposes and not intended to limit the scope of the invention, which will be defined by claims as interpreted according to the principles of patent law, including the doctrine of equivalents.

What is claimed is:

1. A foam medical patch comprising:
a foam substrate having integrated therein an antimicrobial material comprising a silane quaternary ammonium salt.

2. The foam medical patch of claim 1, wherein the silane quaternary ammonium salt comprises 3-(trimethoxysilyl) propyldimethyloctadecyl ammonium chloride.

3. The foam medical patch of claim 1, wherein said antimicrobial material is substantially free from arsenic, silver, tin, heavy metals and polychlorinated phenols.

4. A method for making a foam medical patch, the method comprising:
providing a foam slurry including therein an antimicrobial material comprising a silane quaternary ammonium salt; and
processing the slurry to form the foam medical patch.

5. The method of claim 4, wherein the silane quaternary ammonium salt comprises 3-(trimethoxysilyl) propyldimethyloctadecyl ammonium chloride.

6. The method of claim 4, wherein said antimicrobial material is substantially free from arsenic, silver, tin, heavy metals and polychlorinated phenols.

7. A method is provided for making a foam medical patch, the method comprising:
providing a foam medical patch; and
treating the outer surfaces of the foam medical patch with an antimicrobial material comprising a silane quaternary ammonium salt.

8. The method of claim 7, wherein the silane quaternary ammonium salt comprises 3-(trimethoxysilyl) propyldimethyloctadecyl ammonium chloride.

9. The method of claim 7, wherein said antimicrobial material is substantially free from arsenic, silver, tin, heavy metals and polychlorinated phenols.
